(19) United States
(12) Patent Application Publication
(10) Pub. No.: US 2022/0202824 A1
(43) Pub. Date: Jun. 30, 2022

(54) Methods for Treating Centronuclear Myopathy

(71) Applicant: Stemline Therapeutics, Inc., New York, NY (US)

(72) Inventors: Enrique Poradosu, Brookline, MA (US); Thomas Bartholomew Crabbe, Berkshire (GB)

(73) Assignee: Stemline Therapeutics, Inc., New York, NY (US)

(21) Appl. No.: 17/275,293

(22) PCT Filed: Sep. 13, 2019
(86) PCT No.: PCT/US2019/051029

§ 371 Date: Mar. 11, 2021

Related U.S. Application Data
(60) Provisional application No. 62/730,661, filed on Sep. 13, 2018.

(51) Int. Cl. A61K 31/5377

(52) U.S. Cl. CPC A61K 31/5377

(57) ABSTRACT

Disclosed herein are methods for treating centronuclear myopathies, such as myotubular myopathy, in a subject in need thereof, comprising administering to the subject in need thereof an effective amount of 3-{[(3S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxamide and/or a pharmaceutically acceptable form thereof.

### CROSS-REFERENCE TO RELATED APPLICATIONS

This application is a national phase entry pursuant to 35 U.S.C. § 371 of International Application No. PCT/US2019/051029, filed Sep. 13, 2019, which claims priority to U.S. Provisional Application No. 62/730,661, filed Sep. 13, 2018, the contents of each of which are hereby incorporated in their entirety.

### FIELD

The present disclosure relates to compositions and methods for treating centronuclear myopathy using inhibitors of phosphoinositide 3-kinase (PI3K).

### BACKGROUND

Centronuclear Myopathies: Centronuclear myopathies is a category of rare genetic disorders that affect skeletal muscle tissue. A frequent feature of centronuclear myopathies is that the nucleus is often positioned in the center of many of the affected individual's muscle cells, rather than in the normal location at the ends of these cells. The resulting symptoms include extremely low muscle tone that can necessitate assistance for bodily functions such as sitting, eating, and breathing.

One such centronuclear myopathy is myotubular myopathy (MTM). MTM is a rare and severe muscle disorder that occurs with an estimated incidence of 1 male in every 50,000 births. Affected newborn boys, approximately one per 50,000 births, typically display marked hypotonia and respiratory failure (Jungbluth et al., Orphanet J. Rare Dis. 2008, 3, 26). Survival beyond the postnatal period often requires intensive support, such as gastrostomy feeding and/or mechanical ventilation. Currently, there are no established treatments for MTM.

Myotubular myopathy is caused by mutations of MTM1, which encodes a phosphoinositide lipid phosphatase called myotubularin 1 (MTM1). Myotubularin 1 removes the 3-phosphate group from phosphatidylinositol-3-phosphate (PI3P) so a loss of function, as seen in MTM, is predicted to result in increased levels of this phosphoinositide (Pierson et al., Hum. Mol. Genet. 2012, 21(4), 811-825). Observing reciprocal interplay between MTM1 and PI3P kinases, previous studies have suggested that lowering PI3P levels may prevent MTM disease progression (Sabha et al., J. Clin. Invest. 2016, 126(9), 3613-3625).

PI3Ks: Phosphoinositide-3-kinase (PI3K) enzymes are a family of lipid kinases that phosphorylate the 3′ position of the inositol ring on phosphatidylinositol (PI) and higher-phosphorylated polyphosphoinositides. PI3K enzymes are divided into three classes (Classes I, II, and III) based on structure and preferred substrate specificity.

The role of Class I PI3Ks in cellular activation, particularly in lymphocytes, has been examined in considerable detail, and therapeutic approaches based on targeting Class I PI3Ks have been evaluated. In contrast, less is known about the physiological functions of the three monomeric Class II PI3K isoforms, C2A, C2B, and C2G (Harris et al., Mol. Cell. Bio., 2011, 31(1), 63-80). For example, potency on Class I PI3Ks was found not to be an indicator of activity on Class II PI3Ks.

Class I, II, and III PI3Ks: Class I, Class II, and Class III PI3Ks are the kinases generally responsible for generating PI3P. However, PIK3C2B (a Class II kinase), but not PIK3C2A (another Class II kinase), is appreciably expressed in skeletal muscle, the primary tissue affected in MTM. Moreover, PIK3C2B has been identified as a potent genetic modifier of Mtm1 mutation. (Sabha et al., J. Clin. Invest. 2016, 126(9), 3613-3625).

PIK3C2B inhibitors: Therefore, PIK3C2B inhibition has been suggested as a treatment strategy for MTM that may work as a monotherapy or as a complement to other efforts under investigation. At present, there are no reported inhibitors selectively targeting PIK3C2B that have been tested in animal models or in clinical trials.

Moreover, the existing PI3K inhibitors that target PIK3C2B typically have activity against PIK3C3 (Class III) as well. For example, known PI3K inhibitors LY294002, wortmannin, and PI-103, inhibit not only PIK3C2B but also PIK3C3, as well as Class I PI3Ks. Further, the drugs that target Class I or III kinases (but not Class II) have no impact on the mtm zebrafish phenotype. (Sabha et al., J. Clin. Invest. 2016, 126(9), 3613-3625). Therefore, there exists a need for an inhibitor that selectively targets PIK3C2B with relatively less to minimal activity against other PI3Ks for potential treatment of MTM and/or the symptoms thereof.

A series of 6,7-dihydro[1,3]thiazolo[5,4-c]pyridin-4(5H)-one derivatives, which are substituted in the 2-position by a substituted morpholin-4-yl moiety, have been identified as selective inhibitors of PI3K over other human kinases. However, for the treatment of MTM, there is still a need to identify a selective inhibitor of the particular Class II isoform, PIK3C2B, and provide effective treatment methods using the same.

### SUMMARY

In accordance with the description, disclosed herein is a method for treating centronuclear myopathy using a selective inhibitor of human Class II PI3K, PIK3C2B. U.S. Pat. No. 8,242,116 discloses 3-{[(3S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxamide of the following structure:

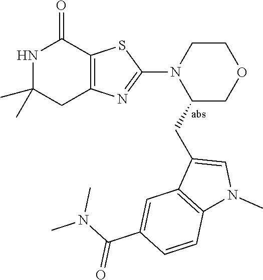

as being a Class I PI3K inhibitor. It has been found, surprisingly, that 3-{[(3S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxamide has a selective binding affinity (IC₅₀) for the human PI3K Class II, PIK3C2B.

Accordingly, one aspect of the present disclosure is a method for treating centronuclear myopathy in a subject in need thereof, comprising administering to the subject in need thereof an effective amount of 3-{[(3S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxamide and/or a pharmaceutically acceptable form thereof.

Another aspect of the present disclosure is a method for inhibiting PIK3C2B kinase in a sample exhibiting organelle mislocalization, comprising administering to the sample an effective amount of 3-{[(3S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxamide and/or a pharmaceutically acceptable form thereof.

Another aspect of the present disclosure is a method for ameliorating the symptoms of centronuclear myopathy in a subject, comprising administering to the subject in need thereof an effective amount of 3-{[(3S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxamide and/or a pharmaceutically acceptable form thereof.

Another aspect of the present disclosure is a method for treating muscle weakness and/or muscle atrophy in a subject in need thereof, comprising: identifying said muscle weakness and/or muscle atrophy (in other words, muscle degradation) as being linked to excess PIK3C2B kinase, and administering an effective amount of 3-{[(3S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxamide and/or a pharmaceutically acceptable form thereof to the subject in need thereof. In another aspect is a method for treating muscle weakness and/or muscle atrophy in a subject in need thereof, comprising: identifying said muscle weakness and/or muscle atrophy (in other words, muscle degradation) as being linked to (a) a loss of MTM1 activity, optionally wherein the loss of MTM1 activity is due to mutations in MTM1, and/or (b) an excess of PI3P, and administering an effective amount of 3-{[(3S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxamide and/or a pharmaceutically acceptable form thereof to the subject in need thereof.

Yet another aspect of the present disclosure is a pharmaceutical composition for treating an adult or pediatric patient in need of inhibition of PIK3C2B kinase comprising 3-{[(3S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxamide and/or a pharmaceutically acceptable form thereof and a pharmaceutically acceptable excipient. In one aspect, the pediatric patient has been diagnosed with centronuclear myopathy.

Additional objects and advantages will be set forth in part in the description which follows, and in part will be understood from the description by one skilled in the art, or may be learned by practice. It is to be understood that both the foregoing general description and the following detailed description are exemplary and explanatory only and are not restrictive of the claims.

The accompanying drawings, which are incorporated in and constitute a part of this specification.

### BRIEF DESCRIPTION OF THE DRAWINGS

FIGS. 1A and 1B show the inhibition profile of PIK3C2B and PIK3C2A (both Class II PI3K), in the presence of the inhibitor compound, 3-{[(3S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxamide, as a plot of % inhibition versus inhibitor concentrations.

### DETAILED DESCRIPTION

As discussed above, the methods of treatment disclosed herein relate to treating centronuclear myopathy in a subject in need thereof comprising administering 3-{[(S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxylic acid dimethylamide and/or a pharmaceutically acceptable form thereof to said subject. In at least one embodiment, the centronuclear myopathy is myotubular myopathy. In at least one embodiment, the centronuclear myopathy is an autosomal myopathy.

Also as discussed above, the methods of treatment disclosed herein relate to treating muscle weakness and/or muscle atrophy in a subject in need thereof, comprising: identifying said muscle weakness and/or muscle atrophy as being linked to excess PIK3C2B kinase, and administering an effective amount of 3-{[(3S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxamide and/or a pharmaceutically acceptable form thereof to the subject in need thereof. In another aspect is a method for treating muscle weakness and/or muscle atrophy in a subject in need thereof, comprising: identifying said muscle weakness and/or muscle atrophy (in other words, muscle degradation) as being linked to (a) a loss of MTM1 activity, optionally wherein the loss of MTM1 activity is due to mutations in MTM1, and/or (b) an excess of PI3P, and administering an effective amount of 3-{[(3S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxamide and/or a pharmaceutically acceptable form thereof to the subject in need thereof. In at least one embodiment, the muscle weakness and/or muscle atrophy (in other words, muscle degradation) is linked to a loss of MTM1 activity. In at least one embodiment, the loss of MTM1 activity is due to at least one mutation in MTM1. In at least one embodiment, the at least one mutation in MTM1 results in a loss of or reduction in function of MTM1. In at least one embodiment, the muscle weakness and/or muscle atrophy (in other words, muscle degradation) is linked to an excess of PI3P. In at least one embodiment of these treating methods, the subject is 40 years old or less. In at least one embodiment, the subject is 20 years old or less. In at least one embodiment, the subject is 15 years old or less. In at least one embodiment, the subject is 10 years old or less. In at least one embodiment, the subject is 12 months old or less. In at least one embodiment, the subject is 6 months old or less. In at least one embodiment, the subject is 1 month old or less. In at least one embodiment, the subject is in utero.

Also as discussed above, the methods disclosed herein relate to ameliorating the symptoms of centronuclear myopathy in a subject, comprising administering an effective amount of 3-{[(3S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxamide and/or a pharmaceutically acceptable form thereof to the subject in need thereof. In at least one embodiment, the centronuclear myopathy is myotubular myopathy. In at least one embodiment, the centronuclear myopathy is an autosomal myopathy.

Also as discussed above, the methods disclosed herein relate to inhibiting PIK3C2B kinase in a sample exhibiting organelle mislocalization, comprising administering to the sample an effective amount of 3-{[(3S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxamide and/or a pharmaceutically acceptable form thereof. In at least one embodiment, the sample contains fish tissue. In at least one embodiment, the sample contains mammal tissue.

In accordance with the description herein, the present disclosure also relates to a pharmaceutical composition for treating adult and/or pediatric subjects or patients in need of inhibition of PIK3C2B kinase, comprising 3-{[(3S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxamide and/or a pharmaceutically acceptable form thereof, and a pharmaceutically acceptable excipient. When the subject or patient in need is pediatric and/or has difficulty swallowing, care should be taken to select the appropriate composition and ingredients from among those known.

For example, pharmaceutical compositions according to the present disclosure, the compositions may preferably take a form suitable for oral, buccal, parenteral, or rectal administration.

For oral administration, the pharmaceutical compositions may take the form of, for example, tablets, lozenges or capsules prepared by conventional means with pharmaceutically acceptable excipients such as binding agents (e.g., pregelatinized maize starch, polyvinylpyrrolidone, or hydroxypropyl methyl cellulose); fillers (e.g., lactose, microcrystalline cellulose, or calcium hydrogen phosphate); lubricants (e.g., magnesium stearate, talc, or silica); disintegrants (e.g., potato starch or sodium glycollate); or wetting agents (e.g., sodium lauryl sulphate). The tablets may be coated by methods well known in the art. Liquid preparations for oral administration may take the form of, for example, solutions, syrups, or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles, or preservatives. The preparations may also contain buffer salts, flavoring agents, coloring agents, or sweetening agents, as appropriate.

Preparations for oral administration may be suitably formulated to give controlled release of the active compound.

For buccal administration, the compositions may take the form of tablets or lozenges formulated in conventional manner.

The composition may be formulated for parenteral administration by injection, e.g., by bolus injection or infusion. Formulations for injection may be presented in unit dosage form, e.g., in glass ampules or multi-dose containers, e.g., glass vials. The compositions for injection may take such forms as suspensions, solutions, or emulsions in oily or aqueous vehicles, and may contain excipients such as suspending, stabilizing, preserving, and/or dispersing agents. Alternatively, the active ingredient may be in powder form for constitution with a suitable vehicle, e.g., sterile pyrogen-free water, before use.

For rectal administration the compositions according to the present disclosure may be conveniently formulated as suppositories. These can be prepared by mixing the active component with a suitable non-irritating excipient which is solid at room temperature but liquid at rectal temperature and so will melt in the rectum to release the active component. Such materials include, for example, cocoa butter, beeswax, and polyethylene glycols.

In some embodiments, the pharmaceutical composition comprises an effective amount of 3-{[(3S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxamide and/or a pharmaceutically acceptable form thereof. The effective amount of 3-{[(3S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxamide (and/or a pharmaceutically acceptable form thereof) required for the subjects of methods described herein will vary depending on the condition of subject and intended method outcome.

With respect to methods for treating myotubular myopathy, the disclosure contemplates all combinations of any of the foregoing aspects and embodiments, as well as combinations with any of the embodiments set forth in the detailed description and examples.

In some aspects, 3-{[(3S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxamide (the compound) has selective binding affinity (IC₅₀) for the human PI3K Class II, PIK3C2B. In some aspects, the compound has selective binding affinity (IC₅₀) for PIK3C2B over human PI3K Class II, PIK3C2A. In some aspects, the compound has selective binding affinity (IC₅₀) for PIK3C2B over human Class II, PIK3C2G. In some aspects, the compound has selective binding affinity (IC₅₀) for PIK3C2B over human Class I and/or Class III PI3 kinases, such as PIK3C3. In some aspects, the compound has selective binding affinity (IC₅₀) for PIK3C2B over a human kinase that is not a PI3 kinase. Selective binding affinity (IC₅₀) as used herein indicates the compound displays a better binding affinity (lower IC₅₀) for PIK3C2B than the binding affinity of the compound for a comparator enzyme. In some aspects, the binding affinity (IC₅₀) of the compound for PIK3C2B is 1-fold, 2-fold, 3-fold, 5-fold, 10-fold, 100-fold, 200-fold, or 300-fold lower than the binding affinity (IC₅₀) for the comparator enzyme.

### EXAMPLES

### Example 1. Preparation of 3-{[(3S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxamide

The title compound was prepared as described in Example 3 of U.S. Pat. No. 8,710,054 (and also described in Example 127 of U.S. Pat. No. 8,242,116). The entire content of each example is incorporated herein by reference.

### Example 2. Biochemical Assays for PIK3C2B

Initially, 3-{[(3S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxamide (referred as Test Compound here in Example 2) was screened against kinase targets representing diverse protein kinase family members for competitive binding using a commercially available profiling assay of 395 kinases (Ambit Biosciences, KINOMEscan), and the compound was found most likely to inhibit the kinase activity of PI3Ks compared to other kinases. The screen revealed no hits other than PI3Ks at a single concentration of the compound (10 μM).

Kinase activities of PIK3C2B (and PIK3C2A for comparison) were obtained using the SelectScreen™ Profiling Service (Invitrogen) based on Adapta™ universal kinase assay protocol and conditions (Invitrogen Corporation, Cat. No. PV5099).

Adapta™ universal kinase assay: The assay itself can be divided into two phases: a kinase reaction phase, and an ADP detection phase. In the kinase reaction phase, all components required for the kinase reaction are added to the well, and the reaction is allowed to incubate for 60 minutes. After the reaction, a detection solution consisting of a europium labeled anti-ADP antibody, an Alexa Fluor™ 647 labeled ADP tracer, and EDTA (to stop the kinase reaction) is added to the assay well. ADP formed by the kinase reaction (in the absence of an inhibitor) displaces the Alexa Fluor® 647 labeled ADP tracer from the antibody, resulting in a decrease in the TR-FRET signal. In the presence of an inhibitor, the amount of ADP formed by the kinase reaction is reduced, and the resulting intact antibody-tracer interaction results in a high TR-FRET signal. ADP formation is determined by calculating the emission ratio from the assay well.

The emission ratio is calculated by dividing the intensity of the tracer (acceptor) emission by the intensity of the Eu (donor) emission at 615 nm as shown in the equation below. Emission Ratio=AlexaFluor™647 Emission (665 nm)/Europium Emission (615 nm).

Adapta™ Assay Conditions

The Test Compound was screened in 1% DMSO (final solution). For 10-point titrations, 3-fold serial dilutions were conducted from the starting concentrations of 20,000 nM for PIK3C2A and 1,000 nM for PIK3C2B.

Substrate/Kinase Mixtures were prepared as follows:

For PIK3C2A (PI3K-C2 alpha), the 2×PIK3C2A (PI3K-C2 alpha)/PI mixture was prepared in a Kinase Buffer including 00 mM HEPES pH 7.5, 200 mM NaCl, 0.06% CHAPS, 6 mM MgCl2, 2 mM EGTA. The final 10 μL Kinase Reaction has 8 ng PIK3C2A (PI3K-C2 alpha) and 100 μM PI in 57.5 mM HEPES pH 7.5, 100 mM NaCl, 0.03% CHAPS, 3 mM MgCl2, 1 mM EGTA. After the 1 hour Kinase Reaction incubation, 5 μL of Detection Mix was added.

For PIK3C2B (PI3K-C2 beta), the 2×PIK3C2B (PI3K-C2 beta)/PI mixture was prepared in 100 mM HEPES pH 7.5, 200 mM NaCl, 0.06% CHAPS, 6 mM MgCl2, 2 mM EGTA. The final 10 μL Kinase Reaction has 8 ng PIK3C2B (PI3K-C2 beta) and 100 μM PI in 57.5 mM HEPES pH 7.5, 100 mM NaCl, 0.03% CHAPS, 3 mM MgCl2, 1 mM EGTA. After the 1 hour Kinase Reaction incubation, 5 μL of Detection Mix was added.

All ATP Solutions were diluted to a 4× working concentration in water. ATP K_{m (apparent)} was previously determined using a radiometric assay except when no substrate is available in which case an Adapta™ assay was conducted.

The Detection Mix was prepared in TR-FRET Dilution Buffer. The Detection mix consists of EDTA (30 mM), Eu-anti-ADP antibody (30 nM) and ADP tracer. The detection mix contains the EC60 concentration of tracer for 5-100 μM ATP.

Adapta™ assay protocol: 2.5 μL 4× Test Compound or 100 nL 100× plus 2.4 μL kinase buffer was dispensed onto white 384-well small volume polystyrene plate (Corning, Cat. No. 3674). Then 2.5 μL of 4×ATP Solution followed by 5 μL of 2× Substrate/Kinase Mixture were added. The final 10 μL Kinase Reaction has 8 ng PIK3C2B (PI3K-C2 beta) and 100 μM PI in 57.5 mM HEPES pH 7.5, 100 mM NaCl, 0.03% CHAPS, 3 mM MgCl2, 1 mM EGTA After 30 seconds plate shake and 2-minutes centrifugation at 1000×g for 2 minutes, Kinase Reaction incubation was performed at room temperature for 60 minutes.

After the Kinase Reaction incubation, 5 μL of Detection Mix was added. After 30 seconds plate shake and 2-minutes centrifugation at 1000×g for 2 minutes, Detection Mix equilibration incubation was performed at room temperature for 60 minutes.

Adapta™ assay controls: The following controls were made for each individual kinase and were located on the same plate as the kinase. The 0% Conversion and 100% Conversion Controls allow one to estimate the percent ATP Conversion achieved in a specific reaction well. Control wells do not include any kinase inhibitors.

0% Conversion Control (100% Inhibition Control)

The maximum Emission Ratio is established by the 0% Conversion Control (100% Inhibition Control), which contains no ATP in the kinase reaction and therefore exhibits no kinase activity. After addition of the Detection Mix containing EDTA, ATP is added to these wells. ATP addition is required for the 0% conversion controls wells because the ADP antibody binds ATP with low affinity. The ATP in wells with maximum kinase inhibition will displace the ADP tracer slightly, though much less efficiently than ADP.

100% Conversion Control

The 100% Conversion Control wells contain ADP instead of ATP and are designed to allow for the calculation of percent ATP conversion.

0% Inhibition Control

The minimum Emission Ratio in a screen is established by the 0% Inhibition Control, which contains active kinase. This control is designed to produce <40% ATP conversion in the Kinase Reaction. The range of ATP conversion allowed is different for each kinase and set in the linear region.

Known Inhibitor

A known inhibitor control standard curve, 10-point titration, is run for each individual kinase on the same plate as the kinase to ensure the kinase is inhibited within an expected IC₅₀ range previously determined.

Adapta™ data analysis: The fluorescent data obtained was analyzed using a data fitting program, XLfit (ID Business Solutions, Guildford, UK). IC₅₀ values were derived by fitting a sigmoidal dose-response curve to a plot of assay readout over inhibitor concentration.

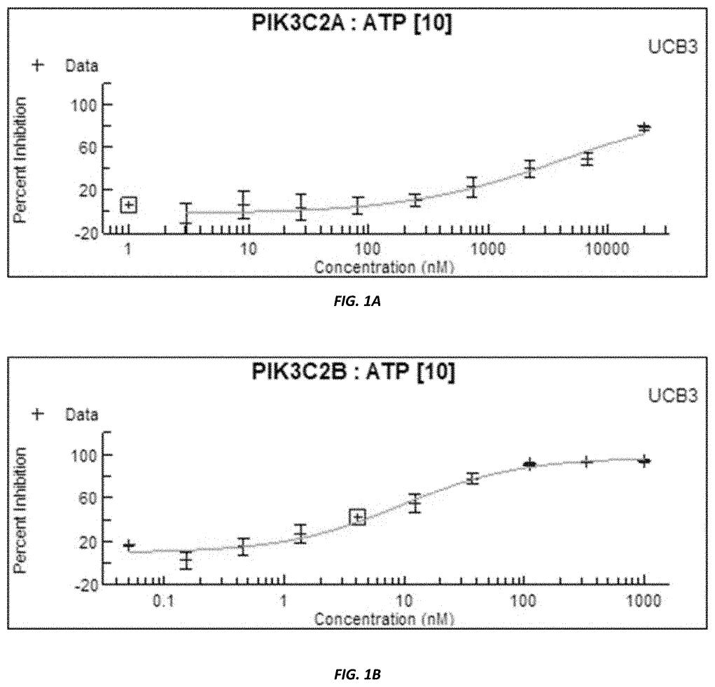

Table 1 shows the inhibition of PIK3C2B and PIK3C2A with the Test Compound, obtained from the above described SelectScreen™ Kinase Profiling Service based on Adapta™ universal kinase assay. FIGS. 1A and 1B show the inhibition curve plotted as % inhibition versus inhibitor concentration for PIK3C2A and PIK3C2B, respectively. The IC₅₀ values for inhibition of PIK3C2A and PIK3C2B with the Test Compound are shown in Table 1. The IC₅₀ for inhibition of PIK3C2B (5.97 nM) was much lower than the IC₅₀ for inhibition of PIK3C2A (4,300 nM), indicating the selective inhibition of PIK3C2B over PIK3C2A.

TABLE 1

| | [ATP] | | Test | | |
|---|---|---|---|---|---|
| Kinase | Tested | IC₅₀ | Compound | % Inhibition | |
| Tested | (uM) | (nM) | (nM) | point 1 | point 2 |
| PIK3C2A | 10 | 4,300 | 20000 | 81 | 78 |
| | | | 6667 | 55 | 46 |
| | | | 2222 | 46 | 36 |
| | | | 741 | 17 | 30 |
| | | | 247 | 15 | 6 |
| | | | 82 | 0 | 11 |
| | | | 27.3 | 13 | −4 |
| | | | 9.1 | −3 | 16 |
| | | | 3.03 | −24 | 3 |
| | | | 1.01 | 25 | −12 |
| PIK3C2B | 10 | 9.57 | 1000 | 93 | 95 |
| | | | 333 | 94 | 93 |
| | | | 111 | 92 | 90 |
| | | | 37 | 74 | 82 |
| | | | 12.3 | 61 | 49 |
| | | | 4.1 | 58 | 27 |
| | | | 1.37 | 21 | 33 |
| | | | 0.46 | 10 | 21 |
| | | | 0.153 | −3 | 8 |
| | | | 0.051 | 16 | 17 |

The above discussed differential inhibition activity of the Test Compound on PIK3C2A versus PIK3C2B was not known and was unexpected. Given that PIK3C2α-deficient mice developed chronic renal failure and exhibited a range of kidney lesions (Harris et al., supra), this finding is an important feature from a safety/toxicity perspective potentially enabling an effective therapeutic window.

Table 2 shows the summary of enzyme inhibition profile of the Test Compound (represented by in vitro IC₅₀) in the present application and previous reports where other known inhibitors of PI3Ks (panPI3K or isoform selective PI3K Class I inhibitors). The differences in IC₅₀ values between PIK3C2B and Class I PIK3s ((PI3Kα, PI3Kβ, PI3Kδ, PI3Kγ) indicate that the inhibition activity on Class I is not a predictor of inhibition activity on Class II, PIK3C2B.

TABLE 2

| | IC₅₀ for | | IC₅₀ for other | Notes (Source of |
|---|---|---|---|---|
| Compound | PIK3C2B | | PI3Ks | reported IC₅₀) |
| 3-{[(3S)-4-(6,6-dimethyl-4- | 9.57 | nM | PIK3C2A: 4,300 nM | 449-fold selectivity over |
| oxo-4,5,6,7- | | | | C2A (Class II isoform) |
| tetrahydro[1,3]thiazolo[5,4- | | | | (Present disclosure) |
| c]pyridin-2-yl)morpholin-3- | | | | |
| yl]methyl}-N,N,1-trimethyl- | | | | |
| 1H-indole-5-carboxamide | | | | |
| (Test Compound) | | | | |
| 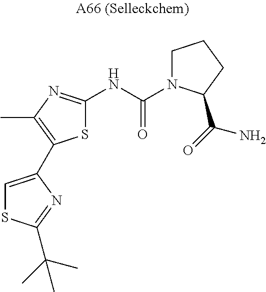 [SMILES: Cc1nc(NC(=O)N2CCC[C@H]2C(N)=O)sc1-c1csc(C(C)(C)C)n1] | 462 | nM | PI3Kα: 32 nM | 14.4 fold selectivity in favor of PI3Kα (Class I) (Abu Eid et al. Cancer Res; 77(15) August 1, 2017) |
| 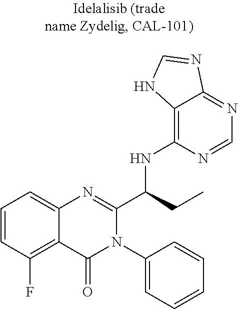 [SMILES: CC[C@H](Nc1ncnc2nc[nH]c12)c1nc2cccc(F)c2c(=O)n1-c1ccccc1] | 1,000 | nM | PI3Kδ: 2.5 nM | 400-fold selectivity in favor of PI3Kδ (Class I) (Kinase Drug Discovery edited by Richard A. Ward, Frederick Goldberg, 2011, RSC publishing) |
| 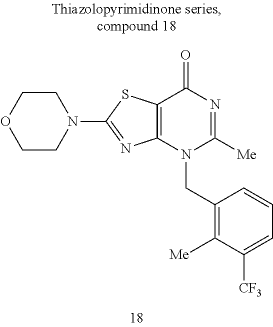 [SMILES: Cc1c(Cn2c(C)nc(=O)c3sc(N4CCOCC4)nc32)cccc1C(F)(F)F] | 1,000 | nM | PI3Kβ: 6 nM | 166-fold selectivity in favor of PI3Kβ (Class I) (Lin et al. ACS Med. Chem. Lett. 2012, 3, 524-529) |
| 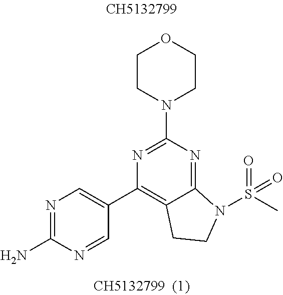 [SMILES: CS(=O)(=O)N1CCc2c(-c3cnc(N)nc3)nc(N3CCOCC3)nc21] | 5,300 | nM | PI3Kα: 14 nM, PI3Kβ: 120 nM PI3Kδ: 500 nM PI3Kγ: 36 nM | Over 10~380-fold selectivity in favor of Class I Kinases (J. Ohwada et al., Bioorg. Med. Chem. Lett. 21 (2011) 1767-1772) |

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

1. A method for treating centronuclear myopathy in a subject in need thereof, comprising administering to the subject an effective amount of 3-{[(3S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxamide and/or a pharmaceutically acceptable form thereof.

2. The method of claim 1, wherein the centronuclear myopathy is myotubular myopathy.

3. The method of claim 1, wherein the centronuclear myopathy is an autosomal myopathy.

4. A method for inhibiting PIK3C2B kinase in a sample exhibiting organelle mislocalization, comprising administering to the sample an effective amount of 3-{[(3S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxamide and/or a pharmaceutically acceptable form thereof.

5. The method of claim 4, wherein the sample contains fish tissue.

6. The method of claim 4, wherein the sample contains mammal tissue.

7. The method of claim 6, wherein the sample contains human tissue.

8. The method of claim 1, wherein treating centronuclear myopathy comprises ameliorating the symptoms of centronuclear myopathy.

9. The method of claim 8, wherein the centronuclear myopathy is myotubular myopathy.

10. The method of claim 8, wherein the centronuclear myopathy is an autosomal myopathy.

11-13. (canceled)

14. A method for treating muscle weakness and/or muscle atrophy in a subject in need thereof, comprising:
a) identifying said muscle weakness and/or muscle atrophy as being linked to (i) excess PIK3C2B kinase, (ii) a loss of MTM1 activity, and/or (iii) an excess of PI3P; and
b) administering an effective amount of 3-{[(3S)-4-(6,6-dimethyl-4-oxo-4,5,6,7-tetrahydro[1,3]thiazolo[5,4-c]pyridin-2-yl) morpholin-3-yl]methyl}-N,N,1-trimethyl-1H-indole-5-carboxamide and/or a pharmaceutically acceptable form thereof to the subject.

15. The method of claim 14, wherein the loss of MTM1 activity is due to mutations in MTM1.

16. The method of claim 14, wherein the subject is 40 years old or less.

17. The method of claim 14, wherein the subject is 20 years old or less.

18. The method of claim 14, wherein the subject is 15 years old or less.

19. The method of claim 14, wherein the subject is 10 years old or less.

20. The method of claim 14, wherein the subject is 1 year old or less.

21. The method of claim 14, wherein the subject is 6 months old or less.

22. The method of claim 14, wherein the subject is 1 month old or less.

23. The method of claim 1, wherein 3-{[(3S)-4-(6,6-dimethyl-4-oxo-4,5,6,7-tetrahydro[1,3]thiazolo[5,4-c]pyridin-2-yl)morpholin-3-yl]methyl}-N,N,1-trimethyl-1H-indole-5-carboxamide has selective binding affinity (IC₅₀) for the human PI3K Class II, PIK3C2B, optionally over one or more of (a) human Class II, PIK3C2A, (b) human Class II, PIK3C2G, (c) a human Class I PI3 kinase, (d) human Class III PI3 kinase, or (e) a human kinase that is not a PI3 kinase.
